(19) United States
(12) Patent Application Publication
(10) Pub. No.: US 2020/0061610 A1
(43) Pub. Date: Feb. 27, 2020

(54) DEVICE, SYSTEM AND METHOD RELATIVE TO THE PRECONCENTRATION OF ANALYTES

(71) Applicant: CENTRE NATIONAL DE LA RECHERCHE SCIENTIFIQUE (CNRS), Paris (FR)

(72) Inventors: Francois-Damien DELAPIERRE, Cachan (FR); Antoine PALLANDRE, Sceaux (FR); Stephane GUILET, Saint-Cheron (FR); Anne-Marie HAGHIRI-GOSNET, Sceaux (FR); Edmond CAMBRIL, Chatenay Malabry (FR)

(21) Appl. No.: 16/348,475

(22) PCT Filed: Nov. 9, 2017
(86) PCT No.: PCT/EP2017/078785

§ 371 Date: May 8, 2019

(30) Foreign Application Priority Data
Nov. 9, 2016 (FR) 1660855

(51) Int. Cl. B01L 3/00; G01N 27/413; G01N 33/487; G01N 27/447

(52) U.S. Cl. CPC B01L 3/502707; G01N 27/413; B01L 2300/0896; G01N 27/44752; B01L 2300/0877; G01N 33/48707

(57) ABSTRACT

The invention proposes several aspects of a Micro-Nano-Micro (MNM) device. According to one aspect, a plurality of observation channels are positioned in parallel, and each has a smaller cross-section in order to generate a disturbance in order to cause preconcentration. The cross-sections of two separate channels are different. According to another aspect, the observation channel has a smaller width, preferably with a constant depth. According to another aspect, a method for analysing and/or distinguishing between and/or sorting analytes is presented. Several types of analytes can be used. According to one aspect, a method for manufacturing a device according to the invention is presented.

### GENERAL TECHNICAL FIELD

The invention concerns the field of devices, systems, and processes for analyzing and/or discriminating and/or sorting particles or molecules present in a solution (more generally referred to as analytes, which may be charged or uncharged, contained in an electrolyte).

The invention also makes it possible to carry out chemical and biochemical reactions by mixing very locally two groups of molecules present in very small quantities.

The invention proposes a solution in the field of microfluidics exploiting the competition of electrophoretic mobility, and advantageously hydrodynamic (additional pressure, Poiseuille flow) and electroosmotic mobility, and more particularly electropreconcentation.

FIG. 1 shows a glass microchannel filled with an ionic solution. Once the pH is higher than about 2, the walls are negatively charged. Consequently, cations (illustrated by “+”) in the bottom electrolyte accumulate near the walls. The cations closest to them cannot move, but the next layers of cations move to a cathode K when an electric field is applied, entraining all the liquid.

This is the electroosmotic flow, which depends on the surface charge of the glass or on the surface zeta potential and which exists even in the absence of analyte in the bottom electrolyte

At the same time, anions (illustrated by “−”) and cations everywhere else in the liquid also migrate to the anode A and the cathode K, respectively.

This is called electrophoretic flow, which depends on the charge/mass ratio of the analyte.

In the case of a microchannel of simple geometry, as shown in FIG. 1 (no restriction, membranes or local modification of the section), these two flows are in opposite directions (see arrows) for anions and in the same direction for cations.

When the migration of some of these ions is disrupted, for example by a restriction of nanometric size, by a porous membrane, or simply by local coating of the walls with a substance that captures certain ions, phenomena of local increases in ionic concentration and, consequently, of rarefaction of ions at other points are observed.

This is called electropreconcentration.

This phenomenon is of great interest from many points of view, it indeed makes it possible to concentrate highly diluted molecules to facilitate their detection and/or to separate ions, which would not concentrate in the same places.

### STATE OF THE ART

The first observations of the phenomenon of ionic preconcentration around a nanoslit were made by Pu et al. in 2004 [Pu-2004] (references are detailed at the end of the description). The device that allowed these observations, represented schematically in FIG. 2, consists of two 100 μm-deep, U-shaped microchannels M1, M2 connected by eight 60 nm-deep nanochannels N0 at the curvature of the U. Each nanochannel forms a nanometric restriction that disrupts anion transfer, which generates the preconcentration phenomenon.

Preconcentration is thus notably a function of the nature and/or geometry of the restriction and the biophysical characteristics of the analyte, such as its mobility.

FIGS. 3a and 3b show the results before and after electropreconcentration. The assembly is filled with a solution containing fluorescein or rhodamine 6G diluted in sodium tetraborate solution (FIG. 3a). When 1 kV electric fields are applied between the anode A and the cathode K, ions concentrate on the cathode side of the device and become scarce on the anode side. This is referred to as concentration polarization (FIG. 3b). An enrichment factor of 100 and a depletion (scarcity) factor of 500 were observed. A simple model based on charge transfer balances and considerations of a double electric layer within the nanochannel was proposed by the authors.

However, the understanding of this phenomenon has been significantly improved by the work of Plecis et al. in 2008 [Plecis-2008]. The device studied this time consists of two microchannels M1, M2 connected by a so-called horizontal-slit nanochannel Nh. This is called a Micro/Nano/Micro (MNM) device. The channels are formed in a plate P extending in a plane XY and are covered by a cover plate C, typically a thin material. Nanochannels extend along the axis X included in the plane XY. A horizontal-slit nanochannel corresponds to a portion of channel whose depth, i.e. the dimension along the axis Z that is orthogonal to the plane XY, is less than the depth of the two microchannels on either side (see FIG. 4).

The simulated solutions consist of fluorescein diluted in KCl solutions of variable ionic strengths.

In the particular case of a glass device, negatively charged on the surface for pH values above 2, anions present in solution, due to wall repulsion, cannot pass through the nanoslit with the same flow rate as cations. The result is an uneven distribution of electrical charges on both sides of the nanoslit. The local electric field therefore varies, and with it the local electrophoretic flow. At certain points, it may happen that the electrophoretic flow compensates for the electroosmotic flow. The velocity of the charged analytes (or ions) is zero. These flow cancellation points (focal points) can be stable (ions/analytes that move away are brought back) or unstable (ions/analytes that move away regain speed and are carried away). Ions/analytes will obviously accumulate at stable focal points.

FIG. 5 illustrates the shape of the total flow (electroosmotic flow+electrophoretic flow) of fluorescein (cᵢₙᵢₜᵢₐₗ=1 nM) according to position in a Micro/Nano/Micro device in a KCL solution (10 μM). H_{micro}=2.5 μm, Hₙₐₙₒ=50 nm. At the point circled in FIG. 5, the velocity of the ions is zero and they accumulate.

An accumulation of analytes in a particular location is referred to as a concentration spot or focal point.

Depending on the value of the electroosmotic flow, this flow profile moves without deformation from top to bottom and this focal point changes position. If the concentration spot is against the nanoslit, it is referred to as “stacking”; if it is far, “focusing”. This stacking and focusing can be anodic or cathodic.

This makes it possible to define four preconcentration regimes: Anodic Stacking (AS), Anodic Focusing (AF), Cathodic Focusing (CF) and Cathodic Stacking (CS).

These results are from [Plecis-2008]. The English terms are established.

A similar horizontal-slit nanochannel Nh device was used by Louër et al. [Louer-2013]. The device integrates a 100 μm-long and 150 nm-deep nanochannel into a 1 μm-deep microchannel (see FIG. 4).

The document WO2010034908 also describes a horizontal nanochannel (see FIG. 10b for example).

Other devices exist to generate the disturbance that causes electropreconcentration.

Sung et al. [Sung-2012] have developed a device based on a channel between two reservoirs, blocked by a porous membrane (Nafion membrane). The channel has a constant width of 50 μm. A variant of the device consists in using different reservoir lengths to observe variations in preconcentration.

The document WO2010052387 presents a device in which the disturbance is generated by a particular polarizable coating.

In terms of protein separation or concentration, the main methods used are chromatography techniques (exclusion, ion exchange, affinity) or gel electrophoresis (Western Blot) or capillary techniques. The separated proteins are analyzed with different means (dichroism, spectrophotometry, NMR, mass spectrometry, X-ray). Immunological methods can also be used (ELISA for example).

Nevertheless, these solutions have several limitations.

The concentration spots generated are not always sufficiently accurate, which prevents the characterization of molecules or particles.

The architecture of the device is not adapted to observe the spots, particularly in a desire to analyze, discriminate or sort particles or molecules.

The horizontal nanoslits have a width that does not favor the development of compact and efficient devices.

Some devices require high electrical voltages, making them unsuitable for the manufacture of efficient and practical devices.

Methods not related to preconcentration are not always fast, efficient and mobile. In particular, surface immunoassays are subject to nonspecific adsorption which sometimes distorts the results.

There is therefore a need to develop a powerful device to discriminate molecules or particles efficiently and quickly.

There is also a need for effective, repeatable, small-scale, etc., means of disturbance to generate electropreconcentration.

### Presentation of the Invention

The invention proposes a device, system and associated process to solve each of the above-mentioned problems.

In a first aspect, the invention concerns a device for the detection/selective preconcentration of charged analytes contained in an electrolyte, comprising:

a plurality of observation channels, each extending in a longitudinal direction in a plane,

two supply channels connected to each other by the plurality of observation channels and configured to convey the electrolyte into said plurality of observation channels, each observation channel being notably formed by:

a nanochannel comprising a first end and a second end, and having a length,

at least one microchannel extending from one of the two supply conduits to one end of the nanochannel, the other end of the nanochannel being connected to the other supply conduit,

wherein the nanochannels have an area section smaller than the section of the at least one microchannel,
wherein, for at least two nanochannels, one dimension among their respective length or their respective section is different.

Advantageously, the device can include the following characteristics, taken alone or in technically possible combination:

the device comprises two microchannels, the first microchannel extending from the first supply conduit to the first end of the nanochannel, the second microchannel extending from the second supply conduit to the second end of the nanochannel,

the device comprising at least three observation channels, wherein each nanochannel has at least one of said dimensions different from the nanochannel of the other observation channels,

the section of a channel defines a width, parallel to the plane and orthogonal to the longitudinal axis, and a depth, orthogonal to the plane and to longitudinal axis, wherein the nanochannels have a width comprised between 50 and 500 nm, and the microchannels have a width comprised between 1 and 20 μm, preferably between 1 and 10 μm,

the section of a channel defines a width, parallel to the plane and orthogonal to the longitudinal axis, and a depth, orthogonal to the plane and longitudinal axis, wherein at least two nanochannels, preferably all, have different widths two to two.

the section of a channel defines a width, parallel to the plane and orthogonal to the longitudinal axis, and a depth, orthogonal to the plane and to the longitudinal axis, wherein the nanochannels have equal lengths.

the section of a channel defines a width, parallel to the plane and orthogonal to the longitudinal axis, and a depth, orthogonal to the plane and to the longitudinal axis, wherein at least two nanochannels, preferably all the nanochannels, have different lengths in pairs,

the lengths of the observation channels are identical,

the microchannels have different lengths,

the section of a channel defines a width, parallel to the plane and orthogonal to the longitudinal axis, and a depth, orthogonal to the plane and to the longitudinal axis, wherein at least one microchannel has a width comprised between 4 and 6 μm, preferably 5 μm, and/or a depth between 0.8 and 1.2 μm,

the observation channels extend in a straight line and parallel to each other,

the device comprises a plate defining the plane, wherein the observation channels are etched on one side of said plate, and comprising a cover plate for covering the observation channels,

for at least one observation channel, the two microchannels of said observation channel have different widths and/or lengths,

In a second aspect, the invention concerns a device for the detection/selective preconcentration or of charged analytes contained in an electrolyte, comprising:

a plate defining a plane,

an observation channel formed in the plate and extending in a longitudinal direction included in the plane,

two supply channels connected to each other by the observation channel and configured to convey the electrolyte the observation channel, the observation channel comprising:
a nanochannel comprising a first end and a second end,
at least one microchannel extending from one of the two supply conduits to a first end of the nanochannel, the other end of the nanochannel being connected to the other supply conduit,
   wherein the width of the nanochannel is less than the width of the microchannel, the width of a channel being the dimension of said channel parallel to the plane and orthogonally to the longitudinal axis.

Advantageously, the device can comprise the following characteristics, taken alone or in technically possible combination:

the device comprises two microchannels, the first microchannel extending from the first supply conduit to the first end of the nanochannel, the second microchannel extending from the second supply conduit to the second end of the nanochannel,

the depth of the nanochannel is equal to the depth of the microchannels, the depth of a channel being the dimension of said channel orthogonally to the plane,

the observation channel is etched on one side of said plate, the device further comprising a cover plate to cover at least said observation channel,

the nanochannel has a width comprised between 50 and 500 nm, and the at least one microchannel has a width comprised between 1 and 20 μm, preferably between 1 and 10 μm,

the device comprises at least two observation channels wherein one observation channel is referred to as primary, another observation channel is referred to as secondary, the two channels being positioned in series between the supply conduits,

the device includes a plurality of observation channels positioned in parallel between the two supply conduits,

the plate comprises a layer of silicon or glass, a layer of silicon dioxide, in which the observation channel is etched,

the two microchannels of an observation channel have different widths and/or lengths.

In a third aspect, the invention concerns a system comprising one of the two devices as described above, and further comprising at least two ports, for fluid communication with the supply channels, and comprising electrodes configured to be placed in the ports and a controllable electrical voltage source capable of generating a potential difference within the observation channels via the electrodes.

The system can further comprise a controllable hydrodynamic pressure generator, associated with at least one of the ports and capable of generating a pressure gradient within the observation channels.

The system further comprises advantageously an imaging device configured to acquire video/images from the observation channels.

In a fourth aspect, the invention concerns a process for the detection/selective preconcentration of charged analytes contained in an electrolyte using one of the two devices as described above or using a system as described above, comprising the following steps:

E1: injection of analytes into the device, so that the observation channels are filled with electrolyte,

E2: application of a voltage at the ends of the observation channels for a specified period of time to cause an electropreconcentration phenomenon and allow analyte concentration spots to appear in the at least one microchannel.

Step E2 may further comprise the application of a pressure gradient within the observation channels for a specified period of time.

Advantageously, the process comprises an additional step E3 of measuring at least one of the following characteristics on each of the microchannels of the different observation channels (for at least two observation channels), for each spot:

longitudinal position in the microchannel of the analyte concentration spot,

size of the spot,

intensity of the spot,

migration velocity of the spot,

width of the spot.

The process can comprise an additional step E4 of comparing the characteristics obtained in step E3 with a database to obtain information about the analyte.

In a fifth aspect, the invention concerns a process for manufacturing one of the two devices as defined above, comprising the following steps:

a preparation step F1 comprising the following steps:
F11: deposition of a silicon dioxide layer on a glass or silicon plate,

an etching step F2 comprising the following steps:
F22: etching the silicon dioxide with fluorine/oxygen/argon plasma.

Advantageously, the device can comprise the following characteristics, taken alone or in technically possible combination:

the process comprises the following additional steps:
the preparation step F1 further comprises:
F13: deposition of an aluminum layer on the SiO₂ layer
F14: deposition of an electron-sensitive resist layer, type ZEP,
F15: lithography to draw the observation channel on the aluminum layer,
the etching step F2 comprises:
F21: etching the aluminum with a chlorine plasma to draw the observation channel on the SiO₂,
a cleaning step F3, comprising the following steps:
F31: Removal of the remaining resist by washing, preferably with acetone and trichloroethylene,
F32: Removal of the remaining aluminum by dissolution, preferably with soda or piranha mixture.

the observation channel etching step F22 consists in etching in a single step the at least one microchannel and the nanochannel, the microchannel (120) having a width greater than the width of the nanochannel (110),

the microchannels have a width comprised between 1 and 20 μm, preferably between 1 and 10 μm, and the nanochannels have a width comprised between 50 nm and 500 nm,

the observation channels are straight,

the silicon dioxide layer etching step F2 is carried out by a jet orthogonal to said layer,

a plurality of observation channels are etched,

the observation channels are spaced in pairs at a distance comprised between 15 and 100 μm, preferably between 30 and 50 μm,

which two supply channels are etched into the silicon dioxide layer to allow the observation channel to be filled with electrolyte.

the process comprises the additional steps F4 of:
F41: covering the glass or silica layer, on the side opposite the silicon dioxide layer, with a resist, preferably AZ5214 resist,
F42: drilling through the glass or silica layer using a mask to form the inlets/outlets of the device, using a microbead jet or a drill,
F43: cleaning with trichoetyhlene, acetone, water, isopropanol and piranha mixture.

the process comprises a step F5 of mounting a thin glass cover plate, to close the channels, preferably by thermal bonding without intermediate resist.

the cover is mounted using an intermediate bonding layer, hydrogen silsequioxane.

### PRESENTATION OF THE FIGURES

Other features, aims and advantages of the invention will emerge from the following description, which is purely illustrative and non-limiting, and which must be read in conjunction with the appended drawings, in which:

FIG. 1, already presented, illustrates the general functioning of flow competition,

FIGS. 2, 3a, 3b, 4, already presented, illustrate devices of the prior art,

FIG. 5, already presented, illustrates the modes of preconcentration,

FIGS. 6 and 7 illustrate a system in which a device in accordance with several embodiments of the invention is positioned,

FIG. 8 illustrates a top view of one embodiment of a so-called vertical-slit device in accordance with the invention,

FIGS. 9a, 9b illustrate two cross-sectional views (longitudinal and orthogonal to each other) of a so-called vertical-slit device in accordance with the invention,

FIGS. 10, 10b illustrate the same two cross-sectional views of a so-called horizontal-slit device of the prior art,

FIG. 11 illustrates one possible embodiment of the device with two observation channels in series, in accordance with the invention,

FIGS. 12, 13, 14a to 14c illustrate a device conforming to a so-called barcode embodiment, in accordance with the invention,

FIGS. 15a to 15c illustrate the preconcentration results in each of the channels of a so-called barcode device, in accordance with the invention,

FIGS. 16a to 16f represent photographs from which the results of FIGS. 15a to 15c were derived,

FIG. 17 illustrates the steps of an analyte detection/analysis/identification process, in accordance with the invention,

FIG. 18 illustrates the difference in the positioning of the concentration spots in the different channels of a so-called barcode device, in accordance with the invention,

FIG. 19 illustrates the underlying physical principle for discriminating between two analytes using a barcode device, in accordance with the invention,

FIG. 20, similar to FIG. 18, illustrates the difference in the positioning of concentration spots in the different channels of a so-called barcode device in accordance with the invention, for two different analytes contained in the same electrolyte,

FIG. 21 illustrates different layers for manufacturing a device in accordance with the invention,

FIG. 22 illustrates the different steps of manufacturing a device in accordance with the invention.

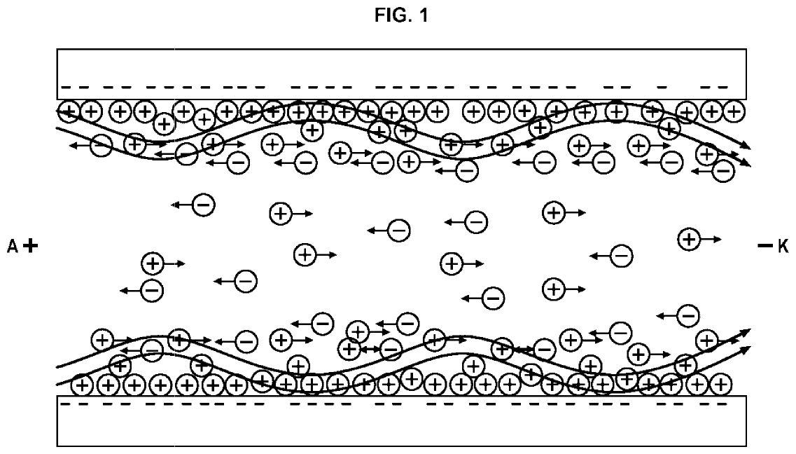

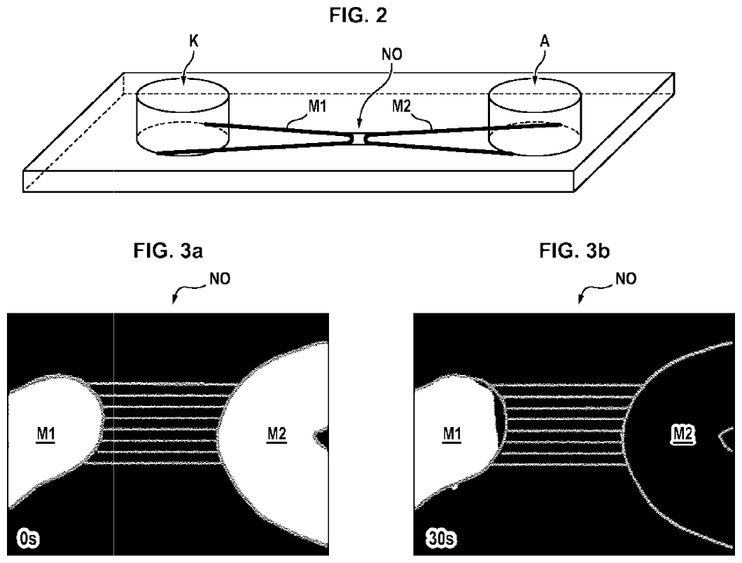

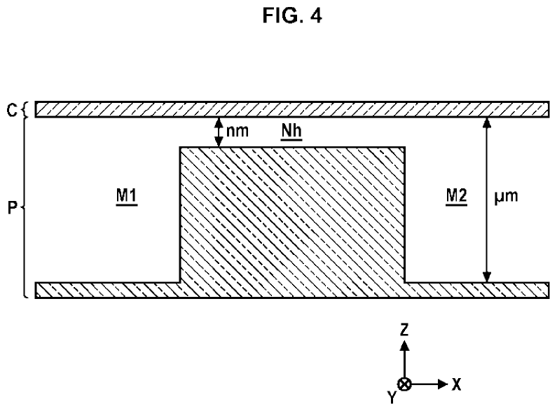

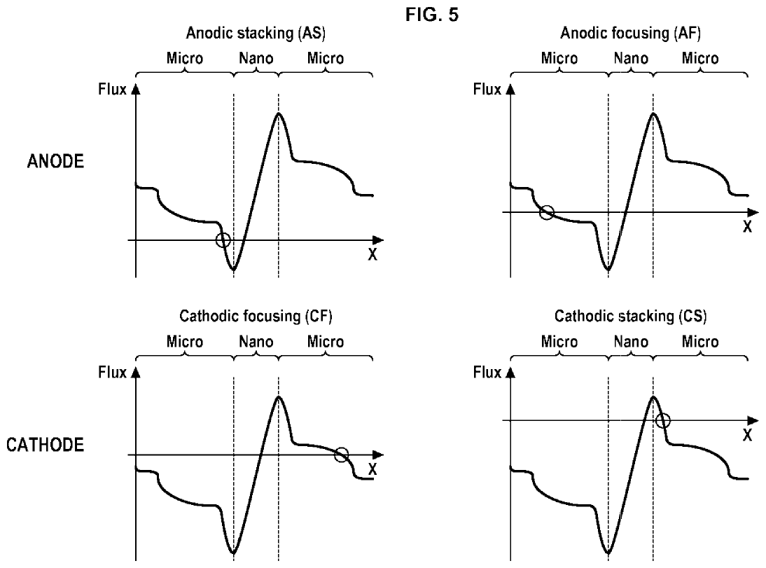

### DETAILED DESCRIPTION

Several aspects of the invention will now be described in detail.

The devices 10 concerned here are devices for the detection/selective preconcentration of charged or uncharged analytes contained in an electrolyte. These devices also allow chemical and biochemical reactions to be carried out very locally by mixing several groups of molecules, generally present in small quantities.

These devices are of the Micro-Nano-Micro (MNM) type, i.e., in the broadest sense, comprising a succession of at least two channels of varying sizes. These devices 10 operate by applying an electrical voltage to its terminals, optionally coupled to a hydrodynamic pressure.

The device 10 is configured to receive electrolytes containing analytes, hereinafter referred to as solution.

### General System

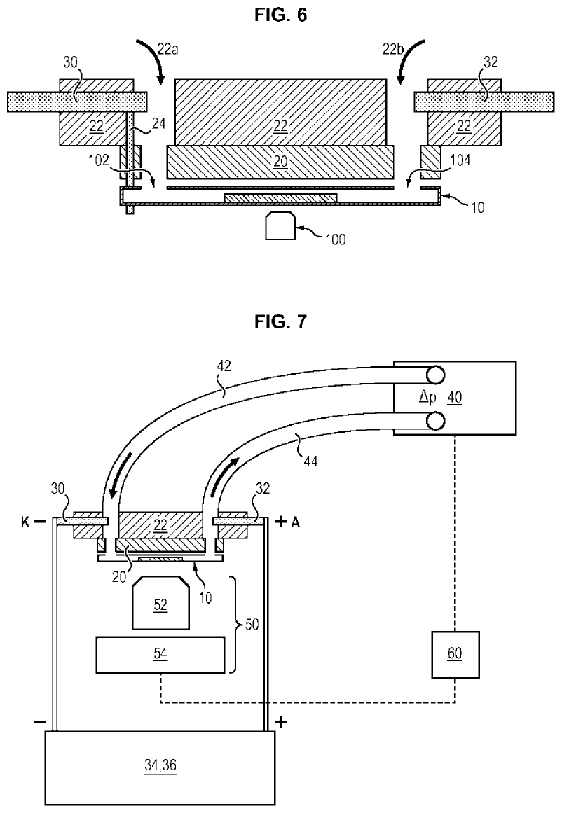

FIGS. 6 and 7 illustrate a general system in which the device is positioned.

The device 10 comprises an observation channel 100, type MNM, within which electropreconcentration phenomena occur. The observation channel 100 extends in a longitudinal direction X. It can be created in different modes, which will be described below.

In use, the observation channel 100 is filled with solution. The device 10 has at least two openings 102, 104, configured to allow a potential difference to be applied in the observation channel 100 using electrodes 30, 32. To that end, each opening 102, 104 is in fluid communication with one of the ends of the observation channel 100.

One embodiment of a system comprises the device 10, a seal 20 typically made of polydimethylsiloxane (PDMS), a block 22 typically made of polymethyl methacrylate (PMMA), assembled one on top of the other and held in place by clamping means 24 such as screws. The PMMA block comprises two ports 22a, 22b opposite the openings 102, 104. The ports 22a, 22b are configured to be filled with solution (for example using a syringe) and are of sufficient size to each accommodate an electrode 30, 32 (for example a platinum electrode). The purpose of the seal 20, positioned between the block 22 and the device 10, is to seal the connection, since the solution level must reach the electrodes which are not in the same plane as the ports 102, 104.

More than two ports 22a, 22b and two openings 102, 104 can be provided, to be able to multiply the number of electrodes forming anode and cathode. This allows for faster filling and easier cleaning.

The electrodes 30, 32 are connected to a controllable voltage generator 34 (FIG. 7) that can typically deliver a potential difference ranging from a few dozen volts to several hundred volts. The voltages it must be able to generate will be specified below according to the embodiments of the device 10. In the case of the experiments performed for the present invention, the generator covered a range from −110V to +110V. A certain number of electronic devices 36 can be coupled to the generator to allow better control or measurement of the applied voltages or electrical characteristics of the system (impedance, etc.).

One of the two ports 22a, 22b thus corresponds to the anode A and the other to the cathode K.

Complementarily, the ports 22a, 22b, or at least one of them, are configured to receive a controllable pressure generator 40. This generator 40 allows a hydrodynamic pressure difference to be applied along the observation channel 100. For example, the pressure controller 40 can be connected to flexible tubes 42, 44 that can be inserted into or encircle the ports 22a, 22b may be suitable.

The pressure values are determined together with the electrical voltage values. Nevertheless, pressures below 1 bar are suitable for moving the spots and making them readable.

An imaging device 50 is provided in the system, to acquire video/images from the observation channel 100. The device 50 generally comprises a microscope 52 coupled to a camera 54. Depending on the wavelengths, visible or not, to be observed, filters can be used and the lighting can be adapted. For example, a mercury lamp with filters can be used to excite and observe fluorescent analytes (for wavelengths in the visible therefore). The imaging device 50 must be able to acquire measurements of low fluorescence intensity to monitor analyte concentration. The microscope 52 is usually coupled with a shutter (Uniblitz VCMD1, for example) to limit photobleaching. One method of rapid identification is to optically read the fluorescence.

The control of the voltage generator 34 and, if need be, the pressure generator 40 is carried out by a calculation unit 60 which can also directly retrieve the images acquired by the imaging device 50.

The observation channel 100 is defined as having a length Lo, along the longitudinal axis X and sections orthogonal to said axis X.

The device 10 consists of three parts in particular: a reservoir part where the solution arrives, an observation zone, connected to the reservoir part, where electropreconcentration occurs, and a disturbance zone, connected to two observation zones on either side of the disturbance zone. The observation zone and the disturbance zone form the observation channel 100. In the case of an MNM device, the observation zone generally has a section whose minimum dimension is of the order of one micrometer, while the disturbance zone has a section whose minimum dimension is of the order of one hundred nanometers. Nevertheless, this generic name MNM cannot be restrictive because it can encompass observation zones whose minimum dimension is of the order of hundred nanometers.

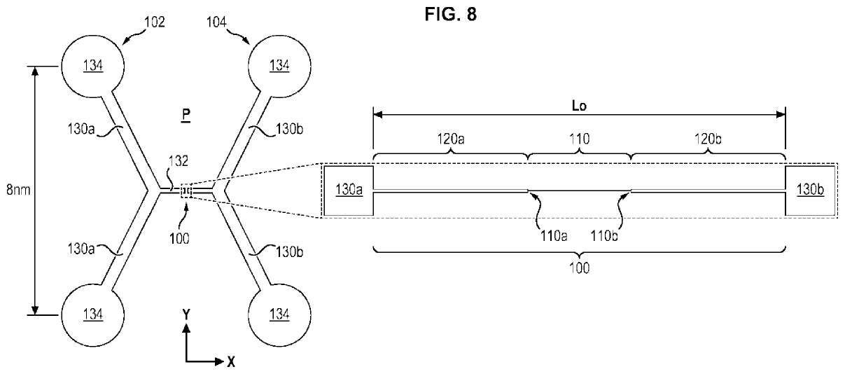

FIG. 8 shows a general embodiment of a device 10.

The device comprises a plate P defining a plane XY. The longitudinal direction X is parallel to this plane XY. One embodiment of the manufacturing of the plate will be detailed below. It is generally made of glass or silica, but not only these.

The observation channel 100 is formed in the plate.

The observation channel 100 comprises a nanochannel 110 with a first end 110a and a second end 110b. The nanochannel 110, as indicated above, forms the disturbance zone.

The observation channel 100 comprises a first microchannel 120a connected to the first end 110a of the nanochannel 110 and a second microchannel 120b connected to the second end 110b of the nanocal 110. The two microchannels 120a, 120b are generically referenced 120.

The three channels 110, 120a, 120b are therefore arranged in series and preferably in a straight arrangement. A total length Lo of the observation channel 100 can then be defined.

Microchannel refers to a channel, i.e. a slit, made in the plate. Micro- and nano- generally and without limitation refer to a minimum dimension of the section, which is either of the order of one micrometer or of the order of one hundred nanometers. More precisely, nano- and micro- mean that a dimension is of the order of a hundred nanometers in one channel and that the same dimension is of the micrometric order in another channel.

A first supply conduit 130a and a second 130b are connected to the first observation channel 110a and the second 110b, respectively. The supply conduits form the above-mentioned reservoir part. Their function is to provide a sufficient volume of solution to allow the proper use of the device 10.

In particular, in a particular geometry, the supply conduits each comprise two separate reservoirs, which meet through an intermediate channel 132 at the end of the first microchannel 120a and the second microchannel 120b, respectively. The dimensions of the sections of the supply conduits are generally greater than 100 μm to provide a sufficiently large amount of solution for preconcentration to occur in the microchannels 120. The supply channels 130a, 130b thus form two symmetrical dihedra transversely to the longitudinal axis X, each dihedron being symmetrical with respect to the longitudinal axis X.

The openings 102, 104 are formed opposite the supply conduits 130a, 130b in general at a zone 134 of greater dimension than the width of the other parts of the supply conduits 130a,130b. In FIG. 8, there are two zones 134 (associated with two openings 102, 102 and 104, 104 and two ports 22a, 22a, and 22b, 22b), symmetrically placed along the longitudinal axis X. By way of example, the two symmetrical zones 134 can be located between 6 and 10 mm apart. In the case of the previous dihedron, the zones 134 are at the ends of said dihedra.

When filled with solution, there is fluid continuity through the supply conduits 130a, 130b (more simply referenced 130) and the observation channel 100.

In a section of a channel, a width I is defined along the axis Y, i.e. the dimension of the channel parallel to the plane XY and orthogonal to the longitudinal axis X, and a depth P is defined along the axis Z, which is the dimension of the channel orthogonally to the longitudinal axis X.

The axes X and Y form a direct angle in the plane XY, and the trihedron XYZ is direct.

The sections of the channels can be rectangular but this involves greater cleaning constraints (particularly because of stagnant zones). Sections with rounded angles provide more stability to the concentration region and improve reproducibility (because it avoids the release effects of stagnant zones). In particular, these so-called rounded sections form transition zones between the microchannels 120 and the nanochannel 110.

A cover plate C covers the channels (observation channel 100 and supply channel 130) to isolate the assembly. As previously mentioned, openings 102, 104 are provided in the plate or cover to allow fluid communication.

To generate a disturbance, the section S110 of the nanochannel 110 has a smaller area than the section S120 of the microchannel 120. Quantities will be explained below.

A Device 100 with a “Vertical-Slit” Observation Channel

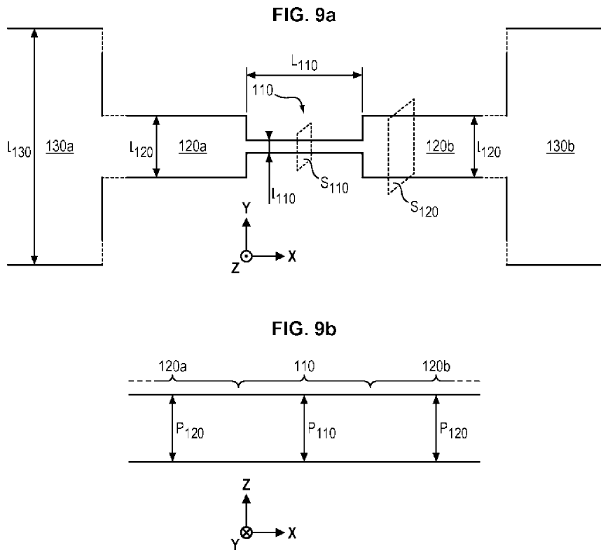

FIGS. 9a and 9b represent one embodiment of a so-called “vertical-slit” device.

The vertical-slit observation channel is defined by the dimensions of the widths of the microchannels 120 and of the nanochannel 110. The width I120 of the microchannels 120 is comprised between 1 and 20 μm, preferably 1 and 10 μm. The width of the nanochannel 110 is comprised between 50 and 500 nm, preferably between 100 and 400 nm. A restriction is then observed in the plane XY, more precisely along the axis Y, which generates a disturbance. To simplify manufacturing, the depth P along the axis Z is constant throughout the observation channel 100, i.e. the depth Pm of the microchannels 120 is the same as the depth P110 of the nanochannel 110.

A depth of around 1 μm is appropriate. Nevertheless, a depth between 100 and 500 nm may also be appropriate, so that the two dimensions of the nanochannel section are of the order of 100 nanometers. In this case, the microchannel 120 has a micrometric dimension and a nanometric dimension and the nanochannel 110 has two nanometric dimensions.

In one embodiment, the width I120 and the length of the first microchannel 120a are equal to the width and length of the second microchannel 120b, respectively. This feature makes it possible to have a symmetrical device, which is important if the desire is to be free from the architectural characteristics of the device 10 to discriminate between two analytes which are each concentrated in one of the microchannels.

In another embodiment, it may be expressly provided to have different widths of microchannels I120. This provides an additional discrimination factor, notably for complex electrolytes, i.e. electrolytes containing a plurality of different analytes with different sizes and chemical groups.

Complementarily or alternatively, the length of the first microchannel 120a may be different from the length of the second microchannel 120b.

The vertical-slit device makes it possible to concentrate analytes by a factor of 1000.

A Device 100 with a “Horizontal-Slit” Observation Channel

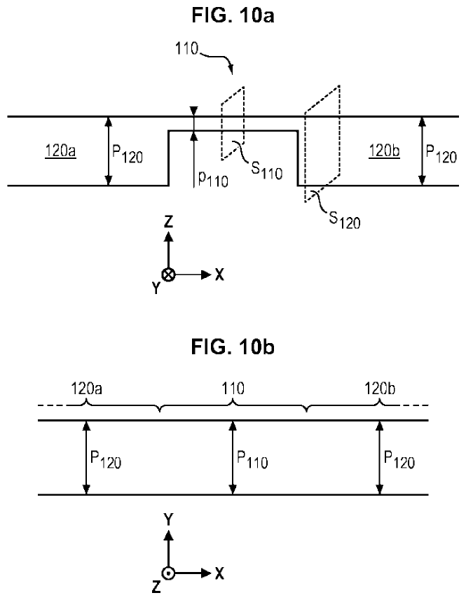

FIGS. 10a and 10b represent one embodiment of a so-called “horizontal-slit” device 100. These devices were presented in the introduction and are already known.

Unlike the vertical-slit observation channel 100, the width of the microchannel I120 is equal to the width along the axis Y of the nanochannel I110. On the other hand, the depth along the axis Z of the nanochannel P110 is less than the depth of the microchannel P120. For example, the depth of the nanochannel P110 is comprised between 75 and 350 nm and the depth of the microchannel P120 is comprised between 2 and 3 μm, for example at about 2.5 μm.

### Comparison Between “Vertical Slits” and “Horizontal Slits”.

Thanks to the “vertical slit”, the preconcentration phenomenon is visible in a direction orthogonal to the nanometric dimension, i.e. the width of the nanochannel 110, easily accessible for the imaging device 50. In addition, the total surface area of the device can be reduced (see in particular the barcode device below), which allows more results to be obtained for the equivalent device size. This allows more results to be obtained for a single experiment, avoiding repeatability contingencies and saving time and material: the ideal parameters can be found in the same operation.

Further about the Microchannels

In a particular embodiment, which applies to any vertical- or horizontal-slit observation channel 100, only one microchannel 120a or 120b is provided. Consequently, the nanochannel 110 is connected on the other side directly to the supply channel 130. The arrangement, in series, is thus as follows: supply channel 130a, microchannel 120a, nanochannel 110, supply channel 130b.

This embodiment, although not symmetrical, can be advantageously used with the application of hydrostatic pressure that allows the concentration spot to be moved.

The present description has been made for two microchannels 120a, 120b but applies to the device with only one microchannel.

A Device 100 with Two Observation Channels in Series.

In one embodiment illustrated in FIG. 11, the observation channel 100 is called primary and the device comprises at least one other observation channel called secondary, located between the primary observation channel 100 and one of the supply channels 120a, 120b, so that the primary 100 and secondary 100′ channels are arranged in series. A buffer zone ZT is provided between the two observation channels 100, 100′.

More than two observation channels 100, 100′ can thus be provided in series.

The primary 100′ and secondary 100′ observation channels may have identical dimensions for reasons of symmetry. Alternatively, having different primary 100 and secondary 100′ observation channels makes it possible to observe more different behaviors for the analytes present in the electrolyte.

Hydrodynamic pressure can also be applied at the zone ZT, replacing or complementing the other application locations described above. The device must then be adapted, particularly in terms of opening, ports and sealing.

Several arrangements of primary and secondary channels 100, 100′ in series can be provided in parallel, by sharing a single supply channel 130a, a single supply channel 130b and, if need be, a single buffer zone. This configuration with several observation channels in parallel is called “barcode”.

### A Barcode Device 10

The “vertical-slit” observation channel is particularly suitable for a so-called “barcode” device 10. Indeed, the latter is easy to manufacture and generates localized spots Sp.

Nevertheless, a horizontal-slit device can also be used.

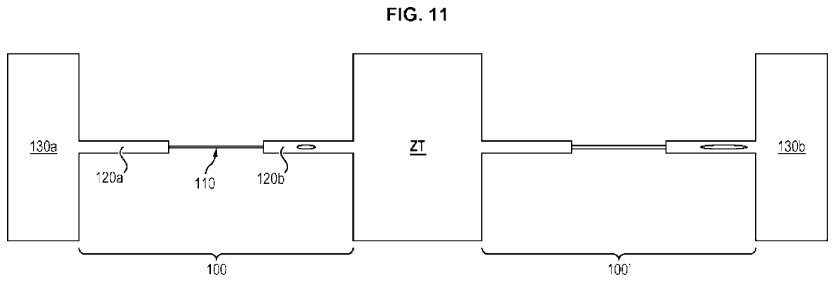

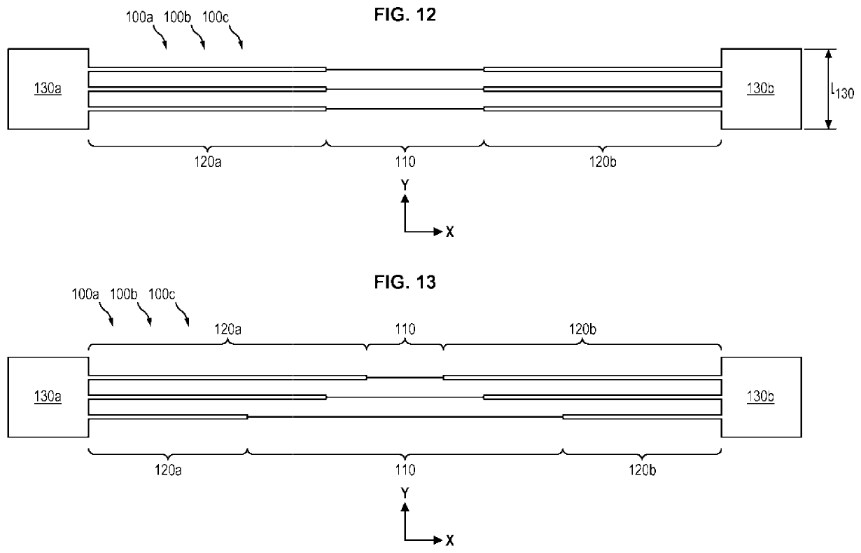

With reference to FIGS. 12 to 13 in particular, the barcode device 10 comprises a plurality of observation channels 100a, 100b, . . . each extending in a longitudinal direction X in a plane XY. Preferably, to simplify measurements, simplify architecture and simplify manufacturing, the observation channels 100a, 100b are parallel to each other.

In this barcode device 10, for at least two nanocals, one dimension among their respective length or their respective section S110 is different. In other words, all the nanochannels 110 do not have the same length or the same section S110.

Preferably, to obtain as many measurements as possible in a single experiment, there is only one nanochannel 110 verifying a given length/section pair, or the number of redundancies, i.e. observation channels with the same characteristics, is limited.

For example, if the barcode device 100 comprises two vertical-slit observation channels 100a, 100b, the following configurations may be available:

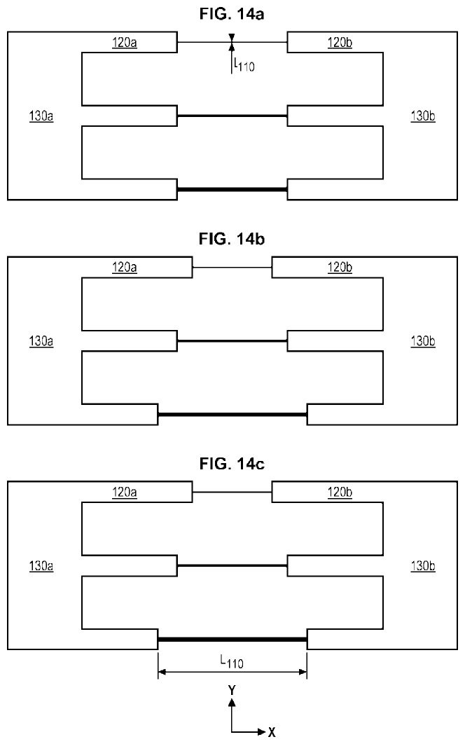

L(100a)=L(100b), I(100a)≠I(100b) (FIG. 14a), or

L(100a)≠L(100b), I(100a)=I(100b) (FIG. 14b), or

L(100a)≠L(100b), I(100a)≠I(100b) (FIG. 14c).

In each of the observation channels 100, and more particularly, each microchannel 120a, 120b of each of the observation channels 100, preconcentration is likely to be observed.

Preferably, the length Lo is the same for all the observation channels 100 in order to simplify the manufacture of the device 100. Nevertheless, if necessary, a condition may be the equal length of the microchannels 120 (especially when the nanochannels 110 have different lengths).

The observation channels are spaced in pairs at a distance comprised between 15 and 100 μm, preferably between 30 and 50 μm. The shorter this distance, the more the device can be compact or integrate a large number of observation channels 100.

The supply channels 130a, 130b play an important role as buffer and reservoir. Indeed, it is important that each observation channel 100 can be considered as independent and that there is no mixing between the microchannels 120a,120b. In other words, this means that the volume of solution present in the supply channels is considered infinite from a theoretical point of view. A value of 100 μm for width 1130 is suitable. A depth equal to the depth of the microchannels 120 is appropriate.

The preconcentration phenomenon depends on several factors, notably including the disturbance generated in the channel. Here it is the nanochannel. By changing the widths (and therefore the section) and/or the length, the preconcentration spots Sp are modified. As the position, motion, width, intensity of each preconcentration spot is information that can help identify the observed analyte, the plurality of observation channels 100a, 100b, 100c allows a multiplicity of information to be acquired in a single experiment.

In particular, the preconcentration, the sorting and the separation of different mixed molecules are facilitated by this barcode device which allows the effects of many parameter sets to be explored simultaneously.

It also makes it possible to quickly find the appropriate parameters to perform an experiment with useful results.

As previously mentioned, analysis of the behaviors of the spots Sp on the different observation channels 100 allows the analyte to be identified.

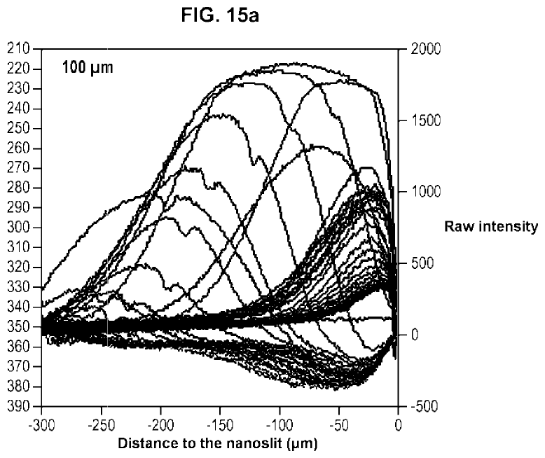

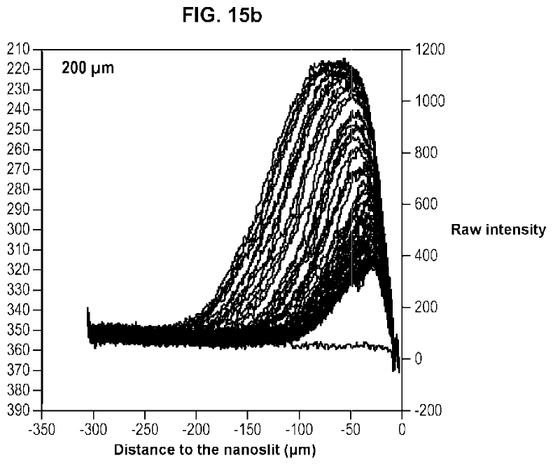

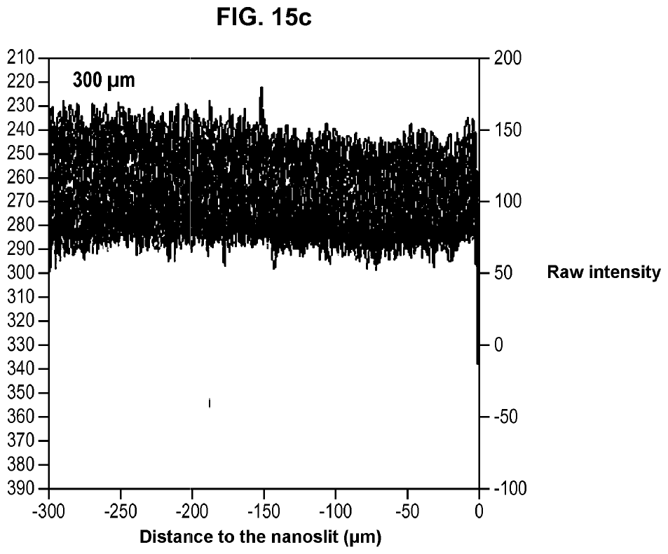

FIGS. 15a, 15b, 15c illustrate the behavior of an analyte in the different observation channels as a function of time, for a variant in accordance with FIG. 14a. Observations include:

for I110=100 nm, an unstable preconcentration, with a spot moving in the microchannel 120 (FIG. 15a),

for I110=200 nm, a stable preconcentration, with a fixed spot (FIG. 15b),

for I110=300 nm, an absence of preconcentration (FIG. 15c).

### Performing an Experiment

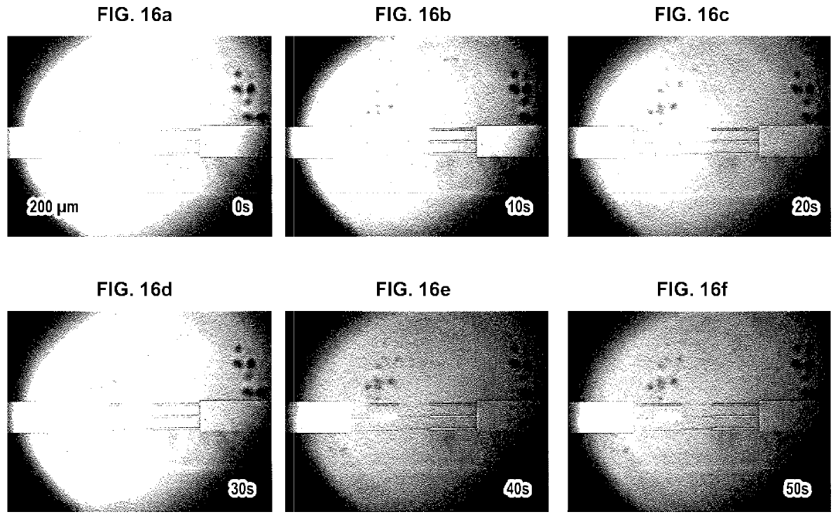

The device 10 is placed within a system as described above. The device is filled through its openings 102, 104, via the ports 22a, 22b, with an ionic solution whose composition or ionic strength can be adapted according to the case. Once the filling is completed, electrical voltages and optionally hydrodynamic pressures are applied within the device. FIGS. 16a to 16f show a preconcentration created with a 10 μM sodium fluorescein solution in a 10 μM KCl solution. This device has three nanoslits of different widths of 100, 200 and 300 nm (from top to bottom in the photos). A voltage of 20 V was applied and a pressure of 0.2 bar in the opposite direction to the electroosmotic flow. The observation time is 50 s. The preconcentration for these parameters is carried out well within the intermediate zone, i.e. in the intermediate microchannels 120 a and 120b, provided for that purpose.

This experiment highlights the advantage of the plurality of slits with different sections, in this case of different width. In a single implementation of the protocol, three preconcentrations are carried out, which makes it possible to deduce which width, taking into account the other parameters, leads to zero or unstable preconcentration, or conversely which width leads to stable preconcentration, which is generally the most easily exploitable data.

By recording the stability and/or associated times and/or position and/or migration velocities and/or spot intensity and/or spot width and/or spot shape, the parameter matrix of the analyte can be formed.

### Role of Pressure

The application of a pressure gradient using the controllable pressure generator 40 allows the preconcentration conditions to be modified.

Indeed, the addition of a pressure flow in addition to the electroosmotic and electrophoretic flows modifies the profile of the total ion flux and the spot moves under its influence. Louër et al. [Louer-2013] have shown that the addition of a pressure flow can stabilize unstable behavior (whose advantage in characterizing an analyte is less important) or change the overall behavior of the observed ion.

It is thus possible to preconcentrate an analyte (BSA in the publication) in a solution (borate buffer) on both the anode and cathode sides depending on the pressure applied.

To identify analytes by observing barcodes consisting of preconcentration spots (or spots) and their behavior, the possibilities offered by the addition of hydrodynamic pressure are interesting since different analytes/ions will not necessarily modify their behavior in the same way depending on the pressure applied. A barcode for each pressure can be observed. Moreover, stable preconcentration is always easier to observe and, as we have seen, pressure is able to stabilize unstable behavior.

### Detection/Selective Preconcentration Process

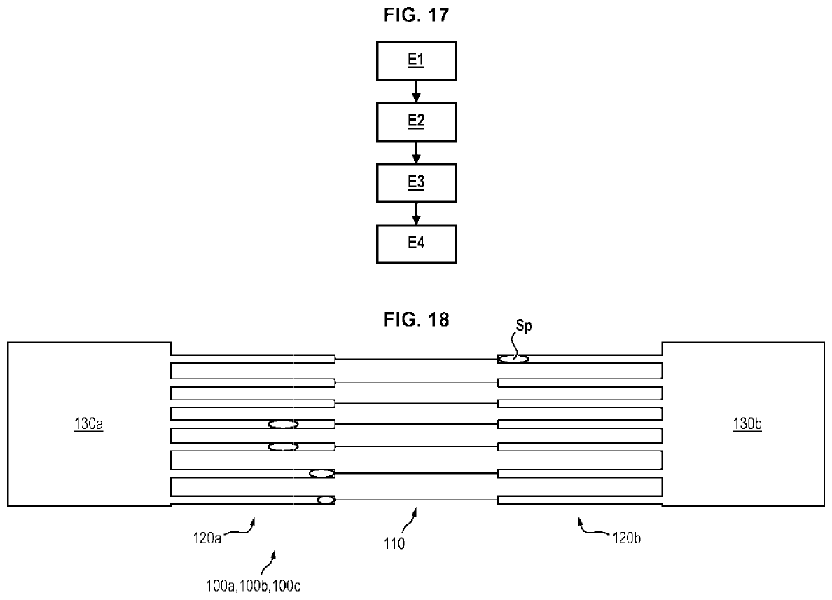

Using the barcode device 100, more precisely using a system comprising such a device, it is possible to implement a process for the detection or identification, or a process for the selective preconcentration, of charged analytes contained in an electrolyte (FIG. 17).

In a first step E1, the supply channels 130, the micronals 120 and the nanochannels 110 are filled with solution. This filling can be done by injection via a syringe.

In a second step E2, electrodes 30, 32 are positioned in the openings 22a, 22b of the system and a voltage is applied using the controllable voltage source 34. The voltage is applied for a period of time specified in the protocol. Under the effect of the voltage, preconcentration phenomena can occur, which generates the appearance of concentration sports of analytes in the microchannels 120.

Complimentarily, for the reasons explained above, a hydrodynamic pressure can be applied on one of the ports 22a, 22b using the controllable pressure generator 40.

In a measurement and analysis step E3, at least one of the following data is acquired for at least two observation channels 100a, 100b (see FIG. 18):

longitudinal position in the microchannel of the concentration spot Sp (or its center, or one of its ends, or both),

size of the spot Sp,

intensity of the spot (in fluorescence for example),

migration velocity of the spot,

width of the spot.

This list is not exhaustive. In particular, the goal is to acquire as many measures as possible. It is also preferable to acquire as much different data as possible from the above data.

Each additional parameter refines the matrix and is therefore able to reduce the set of molecules that can correspond to it.

Preferably, measurements are acquired for each observation channel, the physical characteristics (length, width, depth) of which are known.

In a step E4, the data obtained and/or processed are compared with a database. To that end, the data can be arranged in a matrix for example.

By comparing with a database, for example differences between data, the analyte can be characterized or identified, so that at least one piece of information about the analyte can be obtained. Furthermore, depending on whether hydrodynamic pressure is applied, the mapping may change. A mapping without pressure and a mapping with different pressures can thus be obtained.

The database is generated by calibration on several model solutions containing known analytes.

Thanks to this device, a unique mapping of the analyte is obtained.

Identification with More than One Type of Analyte

In some cases, the solution does not contain a single type of analyte but several. The barcode device 10 makes it possible to select and discriminate these analytes.

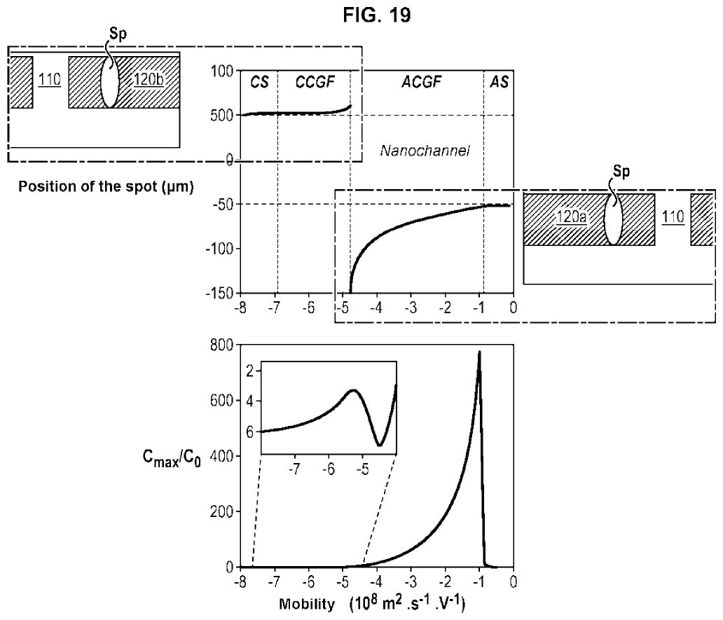

FIG. 19 illustrates the physical basis. Indeed, a molecule or protein with high mobility under electric field tends to concentrate on the cathode side K, while a molecule or protein with low mobility under field tends to preconcentrate on the anode side A. Mention may be made of fluorescein and bovine albumin serum, respectively.

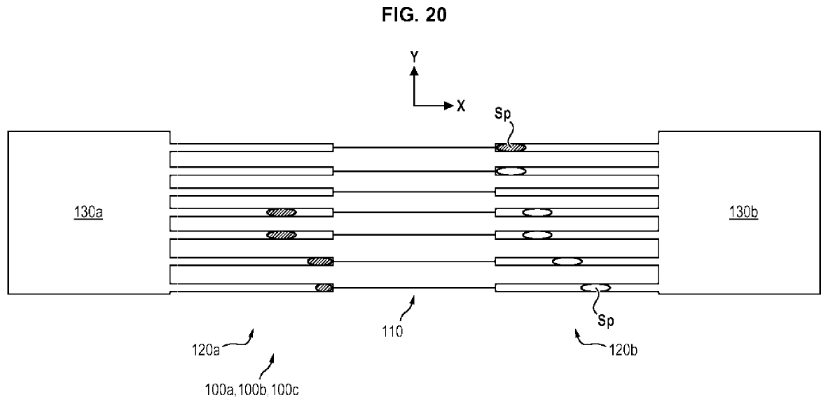

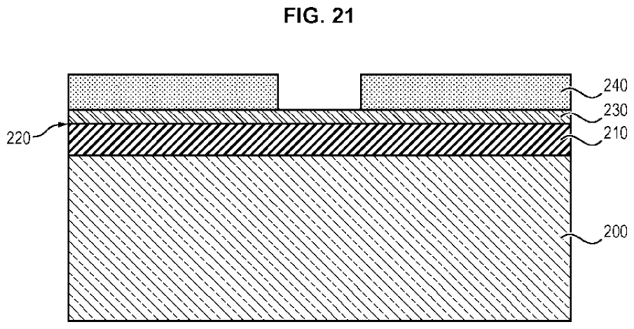

If the mobilities are sufficiently distinct and the spots relative to each type of analyte are distinct, as shown in FIG. 20 (the concentration spots are illustrated by white and black circles, corresponding to the two types of analyte), it is even possible to obtain the matrix of each analyte in a single operation. Depending on the analytes concerned, the controllable pressure generator 40 can then play an important role in making the device 100 effective on analytes.

Thanks to this device, it is possible to identify each existing analyte within a solution containing a complex mixture.

### Details on the Plate of the Device

The plate P of the device (vertical-slit and/or barcode) advantageously comprises a silicon or glass layer and a silicon dioxide SiO₂ layer, in which the observation channel is etched. The SiO₂ layer is purer than silicon or glass and its etching is easier to control than glass.

The cover plate C, usually in the form of a thin material, which covers the channels, can be made of glass.

### Application and Fields

The applications are varied and have been described above. Thanks to its concentration capacity, the barcode device, particularly the vertical-slit device, makes it possible to analyze solutions containing trace or ultra-trace biomolecules, such as threat and biohazard pathogens.

The analytes can be particles, nanoparticles, viruses, proteins, etc.

The fields concerned are scientific research, chemical and bacteriological risk management, pollutant monitoring, drug quality control, detection of disease markers in biological fluids.

### Process for Manufacturing a Vertical-Slit Device

The manufacture of a vertical-slit device 100 requires trenches of varying widths to be etched into a material. This trench must have a controlled width and a relatively large depth to allow for a reasonable flow rate. A depth of 1 μm is suitable, but not limiting. In particular, any depth between 0.8 μm and 5 μm is suitable, and preferably between 1.8 μm and 1.2 μm.

Substrate refers to the assembly of the different layers that are processed to manufacture the device 100 (see FIG. 21). This term is generic and applies to assemblies at any stage.

The described process therefore applies for a device comprising a single vertical slit or a plurality of vertical slits in series, or for a “barcode” device comprising a plurality of vertical slits in parallel and optionally in series.

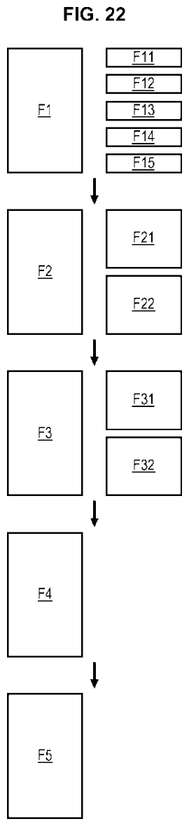

The manufacturing process distinguishes between several main steps (FIG. 22):

a substrate preparation step F1,

an etching step F2,

a cleaning step F3.

Various steps, grouped under the generic term additional steps F4, will also be described. Finally, an assembly step F5 will be described.

In a first step F11, on a glass or silicon plate 200, a silicon dioxide SiO₂ layer, referenced 210, is deposited. The thickness of this layer 210 depends in particular on the depth desired for the observation channel 100. In this case, a thickness of 1 μm is suitable. The layer is deposited by plasma-enhanced chemical vapor deposition (PECVD). The thickness of the glass or silicon plate has no direct impact on the preconcentration. On the other hand, the thickness must be chosen to allow the observation of preconcentration spots (focal length of the lens, absorption of the glass). This plate is also thermalized during subsequent etching, which can change the etching parameters.

Silicon dioxide is softer and purer than silicon and glass. It is this layer 210, therefore, that will be etched.

In a second step F12, a thin layer of germanium 220, of the order of the nanometer, is deposited on the SiO₂.

In a third step F13, a thick layer of aluminum 230 is deposited. Its thickness is comprised between 150 and 250 nm, and is preferably 200 nm. The germanium intermediate layer only reduces the roughness of the aluminum layer.

In a fourth step F14, the aluminum is covered with a layer of positive electron-sensitive resist 240, type “ZEP520A”. This layer 240 has a thickness comprised between 400 and 600 nm, and is preferably 500 nm. This resist is for example deposited by spin coating on the surface at 2000 rpm for 30 s (500 rpm/s acceleration), then annealed 30 min at 160° C.

In a fifth step F15, the electron-sensitive resist is exposed in electron lithography, i.e. electron beam lithography, in order to draw the patterns that will be etched. The resist is developed for 1 min 15 s in a developer (for example ZED-N50). The assembly can be rinsed with isopropanol.

The open patterns in the resist expose the aluminum. The substrate is then ready for the etching step F2.

In a first step F21, the aluminum is etched with chlorine plasma by inductively coupled plasma reactive ion etching (ICP-RIE) to reveal the pattern on the SiO₂. The characteristics used were as follows: ˜35 s, 5 mTorr, Cl2 (20 sccm), bias: −100V, ICP: 500 W. Once the aluminum has been etched by this means, the pattern appears in SiO₂.

In a second step F22, the SiO₂ is then etched with fluorine (SF6)/oxygen/argon plasma. Several parameters can be adjusted in an ICP-RIE plasma: the energy injected into the plasma (ICP) and that transmitted to the ions to bombard the surface (the “bias”), as well as the proportions of the gases. In this case, in one embodiment, the ionic bombardment is activated only part of the time (30-50% over 500 ms), so that the plasma without bombardment, more isotropic, smooths the walls of the etched patterns, and that the charges deposited on the surface by the bombardment are removed. The latter indeed led to a more pronounced etching in the vicinity of the walls. The characteristics used were as follows: ˜13 min, 5 mTorr, SF6 (60 sccm), O2 (3 sccm), Ar (7 sccm), ICP: 300 W, bias: 30 W.

In particular, for the vertical-slit device, the etching step consists in etching all the channels in a single step. The dimensions of the micro- and nanocals have been presented above. In the present case, the etching depth is ideally the same in the micro- and nanochannel.

The etching is stopped before reaching the end of the silicon dioxide layer. Ideally, the etching is configured to stop at the end of the layer. The pattern is then finished. The assembly is then ready for the cleaning step F3.

In a first step F31, the remaining resist is removed by washing, typically with acetone and trichloroethylene.

In a second step F32, the unetched metal, protected by the resist, is removed, typically by dissolution with soda or piranha mixture (H₂SO₄ (50%)/H₂O₂ (50%)).

At the end of these steps F1, F2, F3, a plate cut with trenches that form the channels is obtained.

The additional steps F4 are then provided.

In a first step, the side of the silicon or glass plate opposite the SiO₂ layer is covered with a resist, type AZ5214. The purpose of this layer is to prevent debris from getting trapped in the nanoslit.

In a second step, the layers of the substrate are drilled to generate the openings 102, 104 which allow the injection of solution into the channels. The drilling is done transversely. The openings 102, 104 must therefore open opposite the supply channels. The ports are opened in the substrate with a microbead blaster or a drill. In the case of microbead blasting, a metal guide, i.e. a steel block pierced with holes, is used. These holes are arranged so that they can be aligned with all the ports of the device. The device is bonded to this block, the holes in front of the parts of the pattern to de drilled. A microbead jet is projected from the other side of the guide and erodes the glass or silica at the openings of the metal until it is completely pierced.

In a third step, once the ports have been drilled, the substrate is cleaned with trichloethylene, acetone, water, isopropanol and piranha mixture.

The substrate is then ready to be covered.

In a fifth step F5, the substrate, on the side of the etched channels, is closed with a cover C, typically made of a thin glass. The bonding of this lamella can be done in various ways: thermal bonding (bringing the substrate and the cover into contact, under vacuum, under high pressure and temperature). A preferential embodiment uses a resist, hydrogen silsesquioxane (HSQ), as an intermediate bonding layer using the process described in the document FR 1054183.

The device 10 is then ready to be mounted in the system presented above, allowing the injection of liquid and the application of an electrical voltage.

In order to avoid the widening of the slits during etching, dry plasma etching is preferred to wet etching. The ions constituting the plasma are accelerated with an electric field and bombard the surface to be etched perpendicular thereto. Etching is therefore preferentially done in this direction. Dry etching is therefore likely to present a certain anisotropy. During wet etching, in contrast, chemical reactions occur independently of direction and the substrate is isotropically etched.

Due to the design of the vertical-slit channels, etching is done in only two steps (etching of the metal and etching of the silicon dioxide) that can be performed successively on the same machine.

Conversely, a device that would contain several channels with horizontal slits of different depths would require a mask for each channel depth and therefore a plurality of lithography. Furthermore, the space requirement is larger since horizontal-slit nanochannels have a micrometric dimension in the plane XY. However, a too wide field, for example in the case of acquisition by the imaging device 50 with photodiode, can generate complications.

The manufacturing process involves electron nanolithography and etching.

### REFERENCES

[Pu-2004]: Pu et al. Nano Letters, 2004, DOI: 10.1021/nl0494811
[Plecis-2008]: Plecis et al. Anal. Chem. 2008, DOI: 10.1021/ac8017907
[Louer-2013]: Louër et al. Anal. Chem. 2013, DOI: 10.1021/ac4016159
[Sung-2012]: Ko et al. Lab on a Chip 2012, DOI: 10.1039/c2lc21238b

1. Device (100) for the detection/selective preconcentration of charged analytes contained in an electrolyte, comprising:
a plurality of observation channels (100a, 100b, . . . ) each extending in a longitudinal direction (X) in a plane (XY),
two supply channels (130a, 130b) connected to each other by the plurality of observation channels (100) and configured to convey the electrolyte into said plurality of observation channels (100),
each observation channel (100) being formed by:
a nanochannel (110) comprising a first end (110a) and a second end (110b), and having a length (L110),
at least one microchannel (120ab 120b) extending from one of the two supply conduits (130a, 130b) to one end (110a, 100b) of the nanochannel, the other end of the nanochannel being connected to the other supply conduit,
wherein the nanochannels (110) have an area section (S110) smaller than the section (S120) of the at least one microchannel (120),
wherein, for at least two nanochannels (110), one dimension among their respective length (I110) or their respective section (S110) is different.

2. Device according to claim 1, comprising two microchannels, the first microchannel (120a) extending from the first supply conduit (130a) to the first end (110a) of the nanochannel, the second microchannel (120b) extending from the second supply conduit (130b) to the second end (110b) of the nanochannel (110).

3. Device according to claim 1, comprising at least three observation channels (100a, 100b, 100c), wherein each nanochannel (110) has at least one of said dimensions different from the nanochannel (110) of the other observation channels.

4. Device according to claim 1, wherein the section of a channel defines a width (l), parallel to the plane (XY) and orthogonal to the longitudinal axis (X), and a depth (p), orthogonal to the plane (XY) and to the longitudinal axis (X),
wherein the nanochannels (110) have a width comprised between 50 and 500 nm, and the microchannels (120) have a width comprised between 1 and 20 μm, preferably between 1 and 10 μm.

5. Device according to claim 1, wherein the section of a channel defines a width (1), parallel to the plane (XY) and orthogonal to the longitudinal axis (X), and a depth (p), orthogonal to the plane (XY) and to the longitudinal axis (X),
wherein at least two nanochannels (110), preferably all, have different widths (I110) in pairs.

6. Device according to claim 1, wherein the section of a channel defines a width (l), parallel to the plane (XY) and orthogonal to the longitudinal axis (X), and a depth (p), orthogonal to the plane (XY) and to the longitudinal axis (X),
wherein the nanochannels (110) have equal lengths (L110).

7. Device according to claim 1,
wherein the section of a channel defines a width (1), parallel to the plane (XY) and orthogonal to the longitudinal axis (X), and a depth (p), orthogonal to the plane (XY) and to the longitudinal axis (X),
wherein at least two nanochannels (110), preferably all the nanochannels (110), have different lengths (L110) in pairs.

8. Device according to claim 1, wherein the lengths (Lo) of the observation channels (100) are identical.

9. Device according to claim 2, wherein the microchannels (120a, 120b) have different lengths.

10. Device according to claim 1, wherein the section of a channel defines a width (1), parallel to the plane (XY) and orthogonal to the longitudinal axis (X), and a depth (p), orthogonal to the plane (XY) and to the longitudinal axis (X),
wherein at least one microchannel (110) has a width comprised between 4 and 6 μm and/or a depth comprised between 0.8 and 1.2 μm.

11. Device according to claim 1, wherein the observation channels (100) extend in a straight line and parallel to each other.

12. Device according to claim 1, comprising a plate (P) defining the plane XY wherein the observation channels (100) are etched on one side of said plate (P), and comprising a cover plate (C) for covering the observation channels.

13. Device according to claim 2, wherein, for at least one observation channel, the two microchannels (120a, 120b) of said observation channel (100) have different widths and/or lengths.

14. Device for the detection/selective preconcentration of charged analytes contained in an electrolyte, comprising:
a plate (P) defining a plane (XY),
an observation channel (100, 100a, . . . ) formed in the plate (P) and extending in a longitudinal direction (X) included in the plane (XY),
two supply channels (130a, 130b) connected to each other by the observation channel (100) and configured to convey the electrolyte into the observation channel (100),
the observation channel (100) comprising:
a nanochannel (110) comprising a first end (110a) and a second end (110b),
at least one microchannel (120a, 120a) extending from one of the two supply conduits (130a, 130b) to a first end (110a) of the nanochannel (110), the other end of the nanochannel (110b) being connected to the other supply conduit (130b, 130a),
wherein the width (I110) of the nanochannel (110) is less than the width (I120) of the microchannel (120), the width of a channel being the dimension of said channel parallel to the plane (XY) and orthogonally to the longitudinal axis.

15. Device according to claim 14 comprising two microchannels, the first microchannel (120a) extending from the first supply conduit (130a) to the first end (110a) of the nanochannel (110), the second microchannel (120b) extending from the second supply conduit (130b) to the second end (110a) of the nanochannel (110),

16. Device according to claim 14, wherein the depth of the nanochannel (p110) is equal to the depth of the microchannels (p120), the depth of a channel being the dimension of said channel orthogonally to the plane (XY).

17. Device according to claim 14, wherein the observation channel (110) is etched on one side of said plate (P), the device (100) further comprising a cover plate (C) for covering at least said observation channel (100).

18. Device according to claim 14, wherein the nanochannel (110) has a width comprised between 50 and 500 nm, and the at least one microchannel (120) has a width comprised between 1 and 20 μm, preferably between 1 and 10 μm.

19. Device according to claim 14, comprising at least two observation channels (100, 100′) wherein one observation channel (110) is referred to as primary, another observation channel is referred to as secondary (100′), the two channels (100, 100′) being positioned in series between the supply conduits (130).

20. Device according to claim 14, comprising a plurality of observation channels (100) positioned in parallel between the two supply conduits (130a, 130b).

21. Device according to claim 14, wherein the plate (P) comprises:
a silicon or glass layer (200),
a silicon dioxide layer (210), in which the observation channel (100) is etched.

22. Device according to claim 14, wherein the two microchannels (120a, 120b) of an observation channel (100) have different widths and/or lengths.

23. System comprising a device (100) according to claim 1, and further comprising at least two ports (22a, 22b), for providing fluid communication with the supply channels (130a, 130b), and comprising electrodes (30, 32) configured to be placed in the ports (22a, 22b) and a controllable electrical voltage source (34) capable of generating a potential difference within the observation channels (100) via the electrodes (30, 32).

24. System according to claim 23, further comprising a controllable hydrodynamic pressure generator (40), associated with at least one of the ports (22a, 22b) and capable of generating a pressure gradient within the observation channels (100).

25. System according to claim 23, further comprising an imaging device (50) configured to acquire video/images from the observation channels (100).

26. Process for the detection/selective preconcentration of charged analytes contained in an electrolyte using a device according to claim 1, comprising the following steps:
E1: injection of analytes into the device (100), so that the observation channels (100) are filled with electrolyte,
E2: application of a voltage at the ends of the observation channels (100) for a determined period of time to cause an electropreconcentration phenomenon and to allow the appearance of concentration spots of analytes in the at least one microchannel (120).

27. Process according to claim 26, wherein step E2 further comprises the application of a pressure gradient within the observation channels (100) for a determined period of time.

28. Process according to claim 26 comprising an additional step E3 of measuring at least one following characteristic on each of the microchannels (120) of the different observation channels (100), for each spot (Sp):
longitudinal position in the microchannel (120) of the analyte concentration spot (Sp),
size of the spot,
intensity of the spot,
migration velocity of the spot,
width of the spot.

29. Process according to claim 28, comprising an additional step E4 of comparing the characteristics obtained in step E3 with a database to obtain information related to the analyte.

30. Process for manufacturing a device as defined in claim 1, comprising the following steps:
a preparation step F1 comprising the following steps:
F11: deposition of a silicon dioxide layer (210) on a glass or silicon plate (200),
an etching step F2 comprising the following steps:
F22: etching the silicon dioxide (210) with fluorine/oxygen/argon plasma.

31. Manufacturing process according to claim 30, comprising the following additional steps:
the preparation step F1 further comprises:
F13: deposition of an aluminum layer (230) on the SiO₂ layer
F14: deposition of a layer (240) of electron-sensitive resist, type ZEP,
F15: lithography to draw the observation channel (110) on the aluminum layer,
the etching step F2 comprises:
F21: etching the aluminum (230) with a chlorine plasma to draw the observation channel on the SiO₂,
a cleaning step F3, comprising the following steps:
F31: Removal of the remaining resist (240) by washing, preferably with acetone and trichloroethylene,
F32: Removal of the remaining aluminum (230) by dissolution, preferably with soda or piranha mixture.

32. Manufacturing process according to claim 30, wherein the step F22 of etching the observation channel (100) consists in etching in a single step the at least one microchannel (120) and the nanochannel (110), the microchannel (120) having a width (I120) greater than the width (I110) of the nanochannel (110).

33. Manufacturing process according to claim 30, wherein the microchannels (120) have a width comprised between 1 and 20 μm, preferably between 1 and 10 μm, and the nanochannels (110) have a width comprised between 50 nm and 500 nm.

34. Manufacturing process according to claim 32, wherein the observation channels are straight.

35. Manufacturing process according to claim 30, wherein the step F2 of etching the silicon dioxide layer (200) is done by a jet orthogonal to said layer (200).

36. Manufacturing process according to claim 30, wherein a plurality of observation channels (100a, 100b, . . . ) are etched, each step being repeated as many times as necessary before proceeding to the next step.

37. Manufacturing process according to claim 36, wherein the observation channels (100) are spaced in pairs by a distance comprised between 15 and 100 μm, preferably between 30 and 50 μm.

38. Manufacturing process according to claim 30, wherein two supply channels (130a, 130b) are etched into the silicon dioxide layer (210) to allow the observation channel (100) to be filled with electrolyte.

39. Manufacturing process according to claim 30, comprising additional steps F4 of:
F41: covering the glass or silica layer (200) on the side opposite the silicon dioxide layer (210) with a resist, preferably AZ5214 resist,
F42: drilling through the glass or silica layer using a mask to form the inlets/outlets of the device, using a microbead jet or a drill,
F43: cleaning with trichoethylene, acetone, water, isopropanol and piranha mixture.

40. Manufacturing process according to claim 30, comprising a step F5 of mounting a thin glass cover plate (C) to close the channels (100), preferably by thermal bonding without intermediate resist.

41. Process according to claim 40, wherein the cover (C) is mounted using an intermediate bonding layer, hydrogen silsequioxane (HSQ).

42. System comprising a device (100) according to claim 14, and further comprising at least two ports (22a, 22b), for providing fluid communication with the supply channels (130a, 130b), and comprising electrodes (30, 32) configured to be placed in the ports (22a, 22b) and a controllable electrical voltage source (34) capable of generating a potential difference within the observation channels (100) via the electrodes (30, 32).

43. Process for the detection/selective preconcentration of charged analytes contained in an electrolyte using a device according to claim 14, comprising the following steps:
E1: injection of analytes into the device (100), so that the observation channels (100) are filled with electrolyte,
E2: application of a voltage at the ends of the observation channels (100) for a determined period of time to cause an electropreconcentration phenomenon and to allow the appearance of concentration spots of analytes in the at least one microchannel (120).

44. Process for the detection/selective preconcentration of charged analytes contained in an electrolyte using a system according to claim 23, comprising the following steps:
E1: injection of analytes into the device (100), so that the observation channels (100) are filled with electrolyte,
E2: application of a voltage at the ends of the observation channels (100) for a determined period of time to cause an electropreconcentration phenomenon and to allow the appearance of concentration spots of analytes in the at least one microchannel (120).

45. Process for manufacturing a device as defined in claim 14, comprising the following steps:
a preparation step F1 comprising the following steps:
F11: deposition of a silicon dioxide layer (210) on a glass or silicon plate (200),
an etching step F2 comprising the following steps:
F22: etching the silicon dioxide (210) with fluorine/oxygen/argon plasma.

46. Manufacturing process according to claim 33, wherein the observation channels are straight.
